(19) United States
(12) Patent Application Publication
(10) Pub. No.: US 2019/0083691 A1
(43) Pub. Date: Mar. 21, 2019

(54) UNIT FOR PREPARING AT LEAST ONE SOLUTION OF CONCENTRATES FOR HAEMODIALYSIS AND METHOD FOR PREPARING SAID SOLUTION

(71) Applicant: HEMO PLUS SÀRL, Lausanne (CH)

(72) Inventors: Henri GAUTHIER, Toulouse (FR); Nicolas TILATTI, Boe (FR); Philippe COURTIADE, Aigrefeuille (FR)

(21) Appl. No.: 16/085,718

(22) PCT Filed: Mar. 17, 2017
(86) PCT No.: PCT/CH2017/000029

§ 371 Date: Sep. 17, 2018

(30) Foreign Application Priority Data
Mar. 18, 2016 (CH) 00377/16

(51) Int. Cl. A61M 1/16; A61K 33/14; E04H 1/00; E04H 5/02

(52) U.S. Cl. CPC A61M 1/1656; A61K 33/14; E04H 1/005; A61M 2205/3334; A61M 2205/3393; A61M 2205/70; A61M 2207/10; E04H 5/02

(57) ABSTRACT

A unit (10) for preparing at least one hemodialysis concentrates solution (CH), the solution comprising solid water-soluble materials comprising at least sodium chloride (NaCl), potassium chloride (KCl), calcium chloride (CaCl₂) and magnesium chloride (MgCl₂). This unit has a modular structure and includes the following modules: a sampling module (MPRE) to take at least a sample of each of the raw materials to be dissolved in a volume of purified water, a laboratory module (MLAB) arranged to analyze each of the samples of said raw material to control compliance and quality, a weighing module (MPES) to determine the quantity in weight of the raw material to be introduced in the predetermined volume of purified water, a solution preparation module (MPRE) to introduce and dissolve the quantity in weight of raw material in the predetermined volume of purified water, and at least one module (MCON) for packaging the prepare hemodialysis concentrate solution.

### TECHNICAL SCOPE

This invention relates to a unit for preparing at least one hemodialysis concentrates solution, said solution being composed of solid water-soluble materials comprising at least sodium chloride (NaCl), potassium chloride (KCl), calcium chloride (CaCl₂) and magnesium chloride (MgCl₂), including means for preparing said solution by introducing successively predetermined quantities of said solid materials in a predetermined volume of purified water in a mixing container, means for carrying out individually a dosage control of said solid materials introduced in said predetermined volume of purified water and means for carrying out individually a compliance check of the concentrates solution obtained at the end of the process.

It also relates to a method for preparing at least one hemodialysis concentrates solution, said solution being composed of solid water-soluble materials comprising at least sodium chloride (NaCl), potassium chloride (KCl), calcium chloride (CaCl₂) and magnesium chloride (MgCl₂), wherein one prepares said solution by introducing successively in a mixing container predetermined quantities of said solid materials in a predetermined volume of purified water contained in said mixing container, wherein one carries out individually a dosage control of said solid materials introduced in said predetermined volume of purified water and wherein one carries out individually a compliance check of said concentrates solution for hemodialysis obtained by the method.

### PRIOR ART

The production units for hemodialysis concentrates are generally assembled in industrial production facilities designed to produce important volumes of products that must then be stored, packaged and distributed to the treatment centers. These operations are complicated and expensive, and they imply handling with health and/or medical risks, especially because of a complex packaging management linked with the specific needs of the various treatment centers, the transport and the real-time supply of said care centers.

It appears that the supply of the hemodialysis treatment centers would be significantly simplified, less expensive and less risky if the hemodialysis concentrates production was delocalized and if these products were manufactured on the one hand in an area close to at least one treatment center and preferably at a distance sufficiently small with respect to a group of treatment centers, to be able to guarantee a regular supply on demand, a distribution thanks to which the risks linked with the transport would be reduced, and limited costs to ensure the supply of the treatment centers with the regularity required for this type of treatment.

Article “Unipharm JSC; Products: Concentrates for hemodialysis” describes the preparation protocol of hemodialysis concentrates as it is recommended by the European Pharmacopoeia. According to this protocol, the hemodialysis concentrates are produced in large classical units in the form of very important centralized production facilities, and the hemodialysis concentrates are produced according to methods known per se and which are standardized and implemented in most of the centers manufacturing these compounds. The concentrates are then distributed in the large treatment centers for the preparation of injectable solutions.

### DESCRIPTION OF THE INVENTION

The delocalization of the production of hemodialysis concentrates mentioned above implies the multiplication of production units, these units being of a relatively small size and being located relatively close to the hemodialysis treatment centers.

This is why this invention offers to realize a hemodialysis concentrates production unit with a modular structure, whose operating mode, i. e. the manufacturing method of the products, is identical whatever the unit, so that the design of the units is the same, the manufacturing method of the products, as well as all preparation and component management operations, all maintenance work and all safety actions are identical, in order to guarantee the quality of the manufactured products, optimized production costs and the safety of the supply of the hemodialysis treatment centers.

This goal is achieved by the preparation unit of at least one solution of hemodialysis concentrates according to the invention, characterized in that it comprises the following modules:
a sampling module to take a sample of each of said raw materials to be dissolved in said volume of purified water,
an analysis phase of each of said raw material samples to check their quality,
a weighing module to determine the quantity in weight of said raw materials to be introduced in said predetermined volume of purified water,
a solution preparation module to introduce and dissolve said quantity in weight of said raw materials in said predetermined volume of purified water, and
a module for packaging said hemodialysis concentrates solution obtained.

According to a preferred embodiment, it comprises first analysis means to carry out, during a first intermediate step of said analysis phase of each of said raw material samples, a quality check that consists in checking the compliance of the samples with the theoretical quality defined by at least one certificate specifying the nature and composition of the concerned materials, as well as the compliance with the regulations in force.

According to said preferred embodiment, it also comprises second analysis means to carry out, during a second intermediate step of said analysis phase of each of said raw material samples, a purity check that consists in determining the actual weight of each of said raw materials which corresponds to the weight of the pure active substance contained in these raw materials according to their respective purity.

It moreover advantageously comprises coupling means to interconnect all of the dedicated modules to perform respectively a centralized management of the ambient or purified air flows that circulate through said modules, of the liquid flows entering end exiting the various modules, of the pressurized gas flows, of the electrical energy flows used by the equipment of said modules and of the circulation flows of the workers and of the components used during the method, in the course of said phases of the method and of the intermediate steps.

It preferably comprises a central purification equipment of the atmosphere that circulates through said modules and specific regulation means of the pressure of the air contained in each of said respective modules, means for connecting the modules to said central purification equipment and means for interconnecting the modules to each other and/or to said central purification equipment.

Advantageously, said means for connecting the modules to said central purification equipment comprises an air duct network arranged in a tight space located above a “false ceiling” reserved in the top section of each of said modules, said duct network comprising for each of said modules at least one inlet opening and at least one outlet opening, each of said inlet and outlet openings being equipped with a coupling valve to ensure an interconnection of at least one of said ducts of said duct network.

It preferably comprises a central water treatment equipment that supplies at least a part of said modules, means for distributing the treated water in each of said respective modules, means for collecting the waste water in each of said respective modules and means for connecting the modules to said central water treatment equipment and means for interconnecting the modules to each other and/or to said central water treatment equipment.

In this context, the method according to the invention is characterized in that it comprises the following phases:
a sampling phase to take a sample of each of said raw materials to be dissolved in said volume of purified water,
an analysis phase of each of said raw material samples to check their quality,
a weighing phase to determine the quantity in weight of said raw materials to be introduced in said predetermined volume of purified water,
a solution preparation phase consisting in introducing and dissolving said quantity in weight of said raw materials in said predetermined volume of purified water, and
a phase for packaging said hemodialysis concentrates solution obtained.

According to a preferred embodiment, the analysis phase of each of said raw material samples to check their quality comprises a first intermediate step that consists in checking the compliance of the samples with the theoretical quality defined by at least one certificate specifying the nature and composition of the concerned materials.

The analysis phase of each of said raw material samples to check their quality preferably comprises a second intermediate step including the measurement of the purity of the raw materials, which consists in determining the actual weight of each of said raw materials that corresponds to the weight of the pure active substance contained in these raw materials according to their respective purity.

Said solution preparation phase to introduce and dissolve said quantity in weight of said raw materials in said predetermined volume of purified water advantageously comprises a first intermediate step during which one introduces in said mixing container the quantity in weight of said raw materials required to realize said hemodialysis concentrates solution, said mixing container containing initially a fraction of said predetermined volume of purified water.

Said fraction of said predetermined volume of purified water is advantageously at least approximately comprised between 50 and 80% of said predetermined volume, and preferably close to two thirds of said predetermined volume.

Moreover, said solution preparation phase to introduce and dissolve said quantity in weight of said raw materials in said predetermined volume of purified water can comprise a second intermediate step during which one adds in the mixing container, to the solution obtained after said first step, the remainder of said predetermined volume of purified water.

According to a particularly advantageous embodiment, said packaging phase of said obtained hemodialysis concentrates solution consists in transferring during a first intermediate step said obtained hemodialysis concentrates solution in at least one storage tank.

According to this particularly advantageous embodiment, said packaging phase of said obtained hemodialysis concentrates solution consists in packaging during a second intermediate step said obtained hemodialysis concentrates solution in containers arranged to supply a dialysis generator.

One preferably carries out the phases of the method and of the intermediate steps in dedicated modules, which are interconnected to perform a central management of the ambient air flows that circulate through said modules, of the liquid flows entering and exiting the various modules, of the pressurized gas flows, of the electrical energy flows used by the equipment of said modules and of the circulation flows of the workers and of the components used during the method, in the course of said phases of the method and of the intermediate steps.

In order to perform a central management of the ambient or purified air flows that circulate through said modules, one advantageously arranges an upper zone of each of said concerned modules, in an insulated space that contains circulation paths for an entering atmosphere and an exiting atmosphere, which are respectively in communication with a central air treatment module.

In order to perform a central management of the liquid flows, one advantageously connects the purified water inlets and outlets of the whole of said concerned modules to each other and/or to an inlet and to an outlet of a central water treatment unit.

### BRIEF DESCRIPTION OF THE DRAWINGS

The present invention and its advantages will be better revealed in the following description of an embodiment given as a non limiting example, in reference to the drawings in appendix, in which:

FIG. 1 is a schematic general view that represents the unit for preparing the hemodialysis concentrates produced according to the method of the invention,

FIG. 2 is a detailed view of the weighing module that illustrates the equipment allowing to carry out a preliminary phase for the production of hemodialysis concentrates according to the method of the invention,

FIG. 3 is a detailed view of the preparation module that illustrates the equipment allowing to carry out the actual production of hemodialysis concentrates according to the method of the invention,

FIG. 4 is a detailed view of the storage module that illustrates the equipment allowing to store the hemodialysis concentrates obtained according to the method of the invention,

FIG. 5 is a detailed view of the packaging module of the hemodialysis concentrates obtained according to the method of the invention,

FIG. 6 is a detailed view of the raw material sampling module in order to check their validation,

FIG. 7 is a detailed view of the chemical, physical and bacteriological laboratory module intended to check the quality and purity of the raw materials used in the composition of the hemodialysis concentrates obtained according to the method of the invention, as well as the quality and compliance of the finished or concentrated hemodialysis products,

FIG. 8 is a detailed view of a treatment module for the water used in the composition of the hemodialysis concentrates obtained according to the method of the invention and that is used in the course of the phases of this method,

FIG. 9 is a detailed view of a utilities module that is arranged for treating, preparing and conditioning flows and/or energies used in the course of the method of the invention,

FIG. 10 is a detailed view of a washing module that is arranged for checking the entrance of the operational staff and of the equipment and products used in the course of the method of the invention, and

FIG. 11 is a detailed view of a neutralization module (N.E.U.T) of the effluents produced in the course of the method of the invention.

### BEST WAY OF REALIZING THE INVENTION

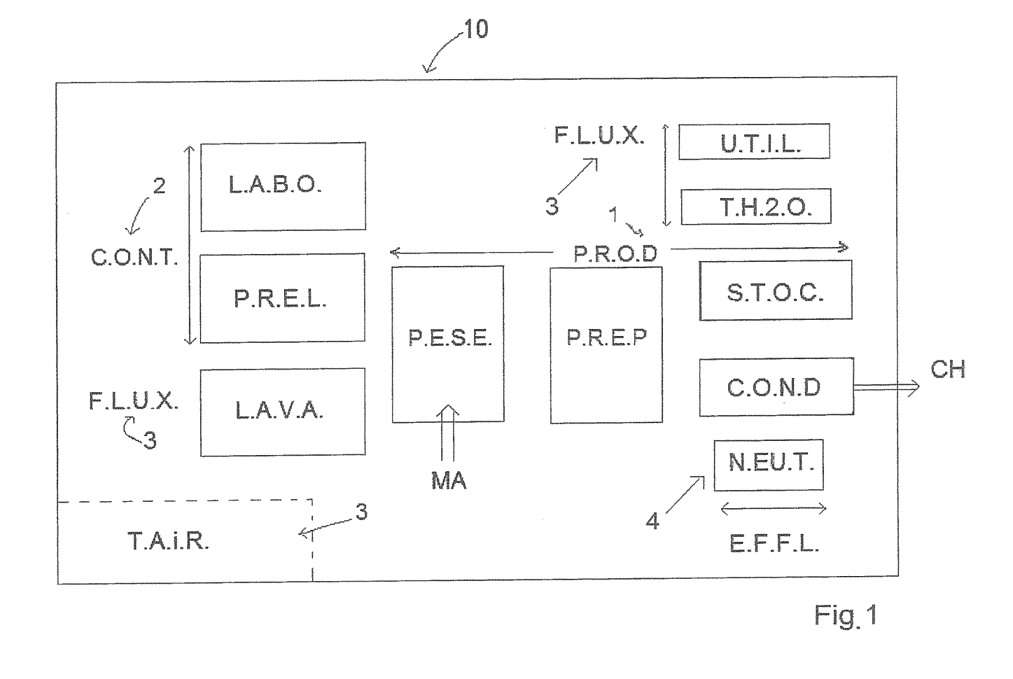

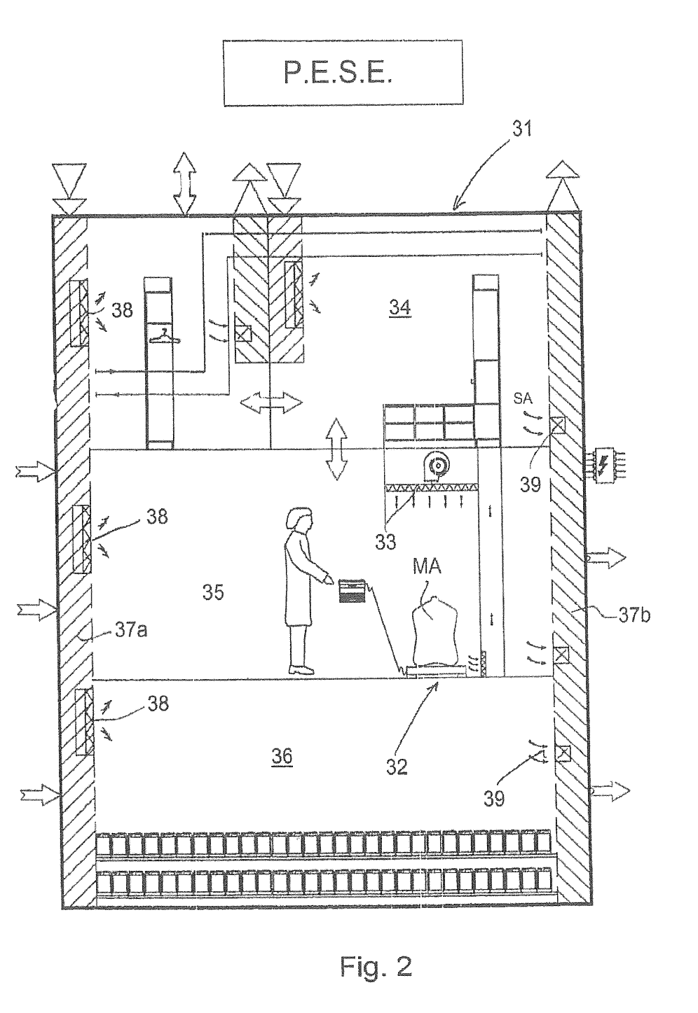

Referring to the figures, in particular to FIG. 1, the represented unit 10 for the production of hemodialysis concentrates (CH) is made of a set of modules, detailed in FIGS. 2 to 9, which are all coupled and interconnected in such a way that the functions of the respective modules and of the equipment they contain can be activated in compliance with the phases of the method according to the invention. Among such modules, one counts a first group 1 of modules called production modules (P.R.O.D) whose function is to produce the (CH) from water-soluble raw materials (MA). Among these modules (P.R.O.D) there is a first module, called weighing module (P.E.S.E) whose function is to prepare the raw material doses that will then be used to produce the (CH) according to the previously elaborated formulations. A second module called preparation module (P.R.E.P) has the function of mixing the solid raw materials in purified water to produce said concentrates. A third module called storage module (S.T.O.C) has the function of collecting in at least one storage tank the concentrates produced by the (P.R.E.P) module in order to contain them temporarily at the end of their manufacture. A fourth module called packaging module (C.O.N.D) has the function of packaging the concentrates in smaller volume containers, which have in particular a sufficient volume to allow, after a suitable final dilution, to carry out a hemodialysis treatment session for a patient. This module could be limited to a packaging machine, without constituting on its own or filling a complex multi-functional module.

It must be noted that the production modules (P.R.O.D) or the specific modules and machines arranged as an addition with respect to the modules are designed to start initially with the sampling of the appropriate raw materials (MA) and to end up with the finished products (CH), ready for distribution, after having undergone all checks, all validations and all safety and quality actions required for a product for medical use as sensitive as hemodialysis concentrates.

Among said modules making up unit 10, one will count a second group 2 of modules called control (C.O.N.T) whose function is to ensure both the check of the raw materials (MA) and that of the finished products, in this case the produced concentrates (CH). A first module called sampling module (P.R.E.L) belongs to this second group, and it consists in taking raw material (MA) samples to check whether they comply with the data declared by the suppliers and with the regulations in force, and with the specificity sheets. A second module called laboratory module (L.A.B.O) belongs to this second group, and it consists in performing chemical, physical and bacteriological analyses of the raw material (MA) samples previously isolated in module (P.R.E.L).

Among said modules making up unit 10, one will count a third group 3 of modules called utilities modules (F.L.U.X), which ensure the preparation and management of the fluids and energies required for the implementation of the method. They contribute to the organization of the circulation of the water flow, of the air flow of the atmosphere in the various modules, of the compressed air flow, of the effluents flow, of the circulation flow of the workers and of the electrical energy supply of the operational components.

The first module of said third group 3, (F.L.U.X) is a so-called water treatment module or (T.H.2.O) module whose function is to prepare the purified water in which the raw materials (MA) will be dissolved to produce the concentrates (CH).

It can for example comprise a reverse osmosis filtration machine or the like in order to remove all solid mineral residues and to neutralize the organic and/or bacteriologic residues.

The second module of this third group 3 is called utilities module (U.T.I.L), and it can in particular group a compressed air generator that supplies the whole of the stations of unit 10 requiring compressed air, a hot water generator, arranged to provide all concerned stations of unit 10 with hot water at the required temperature, a storage tank for the produced hot water and an electric current distributor that ensures the electrical supply of all work stations in the various modules of unit 10.

A third module of this third group 3 of modules is called washing module (LAVA) and is organized to control and manage the workers entrance flow by means of controlled passage systems called locks and of washing equipment.

A fourth module of this third group 3 is called air treatment module (T.A.I.R) and is designed to ensure in particular the dehumidification and purification of the air that circulates in the various modules. To this purpose it comprises an air treatment equipment including a dehumidifier and heating and/or conditioning means known per se, which could be mounted separately outside the other modules.

A fourth group 4 called (E.F.F.L), comprises one or several modules called effluents neutralization module (N.E.U.T), whose function is to treat and if necessary neutralize the effluents generated by the production and the additional treatments, in order to be able to discharge them safety and lawfully at the end of the production of hemodialysis concentrates (CH).

The production of hemodialysis concentrates (CH) begins in first group 1 of modules called production modules (P.R.O.D), and more precisely in weighing module (P.E.S.E) illustrated in FIG. 2. This module 31 is equipped with weighing means 32 arranged underneath an extraction hood 33. An area 34 allows controlling the secured entrance of the staff, an area 35 allows weighing the raw materials (MA) and an area 36 allows packaging the weighed products. The weighing module comprises integrated ventilation means, which are arranged laterally and are made of two channels 37a and 37b running along the side walls of the module. These channels communicate on the one hand with a general aeration and ventilation network that supplies all modules and, on the other hand, with the inside of all areas of the module, according to the specific needs, through wall-mounted flaps 38 and exhausts 39. Channels 37a and 37b are housed in the upper section of module 31, for example in a space separated from the inside of the module by a false ceiling or the like.

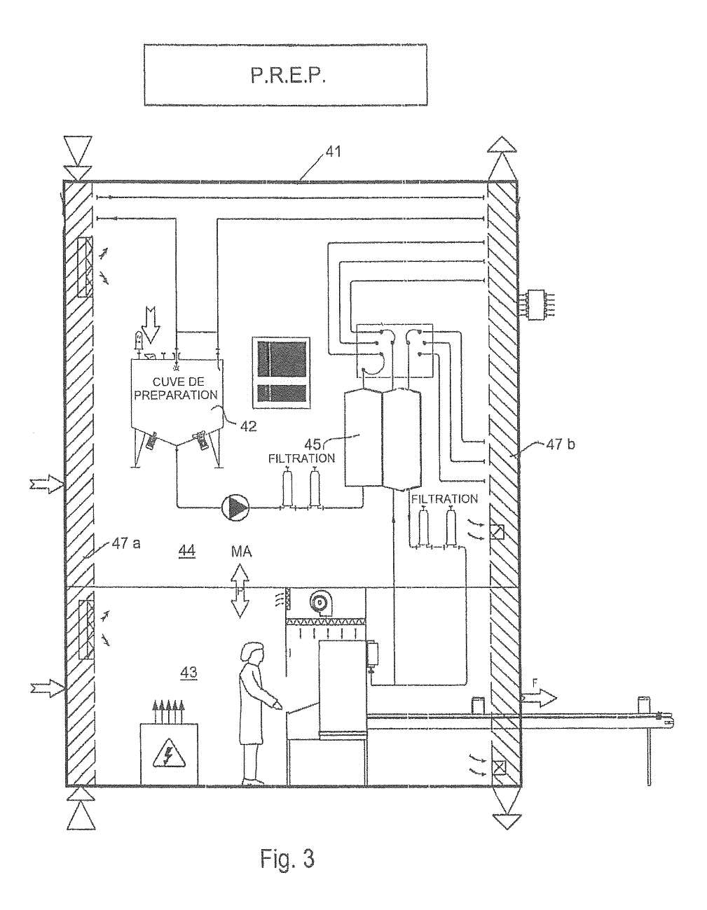

After weighing module (P.E.S.E) illustrated in FIG. 2 comes preparation module 41 (P.R.E.P), illustrated in FIG. 3, whose function is to dissolve the previously dosed raw materials (MA) in purified water. To this purpose, one introduces in a preparation container 42 or mixing container that contains a fraction of the volume intended for producing the planned volume of hemodialysis concentrates (CH) in the course of a first step, then, after stirring this mixture, one completes the volume of purified water to obtain the required result. The raw materials (MA) are brought from an area 43 to an area 44 in which the purified water is introduced in preparation container 41. As for the weighing module illustrated in FIG. 2, preparation module 41 (P.R.E.P) comprises integrated ventilation means, which are arranged laterally and are made of two channels 47a and 47b running along the side walls of the module. These channels communicate with the whole of the ventilation and air distribution circuit and comprise means for intercommunication with the other channels having the same functions of the other modules of unit 10.

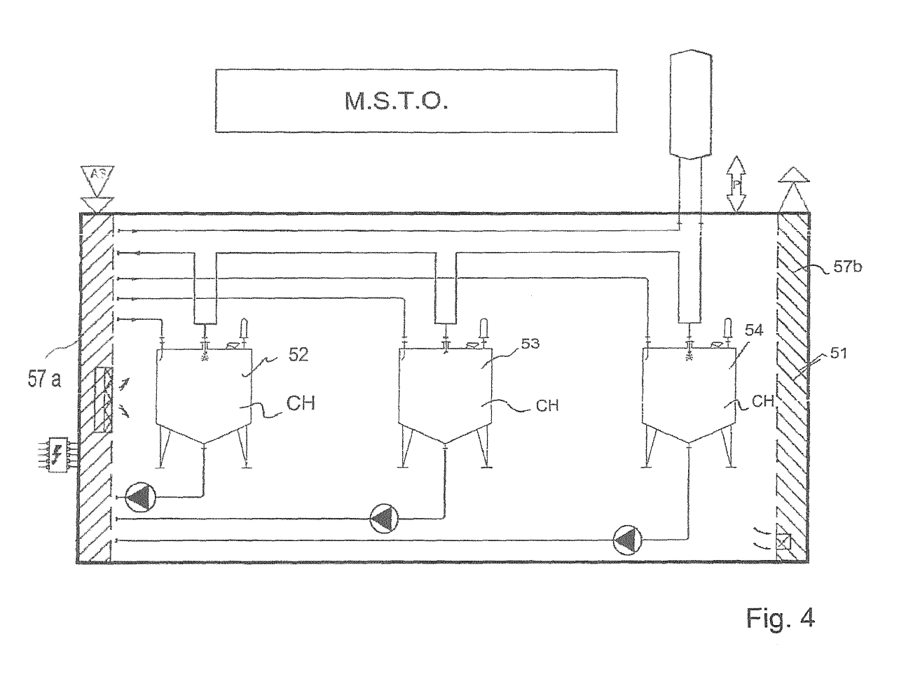

After weighing module (P.E.S.E) illustrated in FIG. 2 and preparation module 41 (P.R.E.P) illustrated in FIG. 3, comes storage module 51 (M.S.T.O), illustrated in FIG. 4, whose function is to store the hemodialysis concentrates (CH) produced, for example in three storage tanks 52, 53 and 54. The storage of the products in these tanks is temporary, and it will be used to supply containers or packagings intended for direct use with patients at the time of a hemodialysis treatment. As for the previous modules of the same production group (P.R.O.D), storage module 51 (M.S.T.O) is equipped with two channels 57a and 57b, which run along the side walls of the module and communicate with the whole of the ventilation and air distribution circuit and comprise means for intercommunication with the other channels having the same functions of the other modules of unit 10.

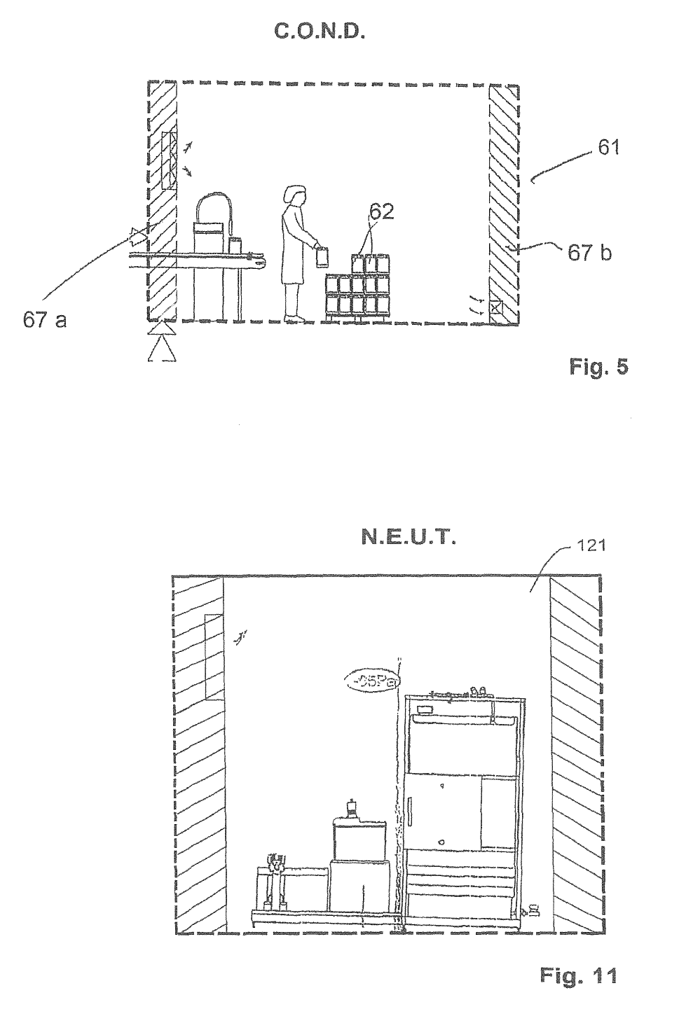

After storage module 51 (M.S.T.O) comes packaging module 61, illustrated in FIG. 5, whose function is to distribute the hemodialysis concentrates (CH) in containers 62 suitable for patients treatment. The produced concentrates (CH) are initially diluted, for example of the order of ten times, to allow their direct consumption by the dialysis machines. Packaging module 61 (C.O.N.D) is equipped with two channels 67a and 67b, which run along the side walls of the module and communicate with the whole of the ventilation and air distribution circuit and comprise means for intercommunication with the other channels having the same functions of the other modules of unit 10.

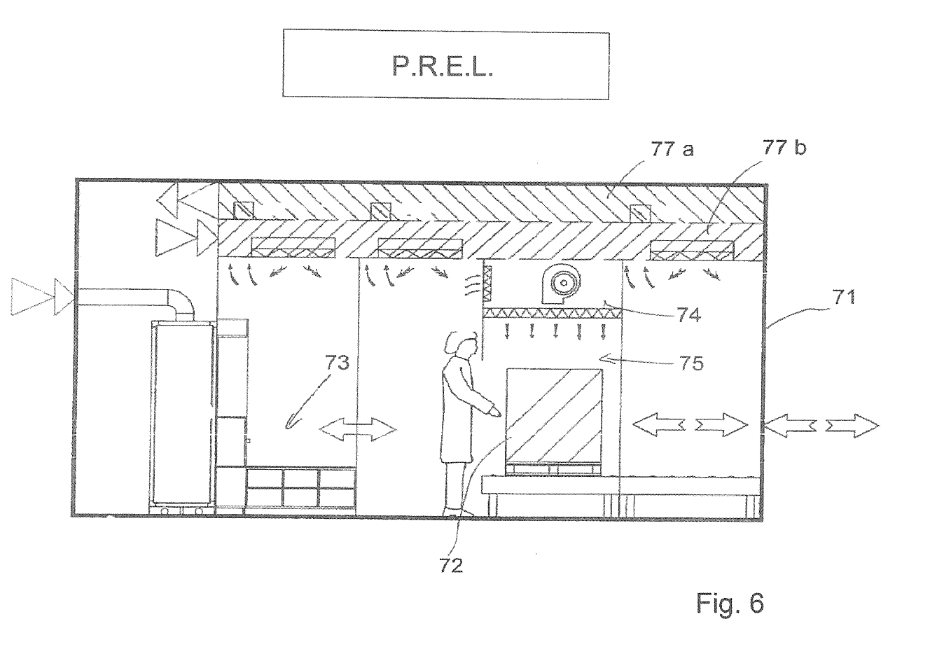

On the other hand, unit 10 comprises a second modules group 2 called control modules (C.O.N.T), in particular a first module 71 called sampling module (P.R.E.L) illustrated in FIG. 6, whose function is to take raw materials (MA) samples in order to check whether they comply with the data declared by the suppliers and with the specificity sheets. Module 71 includes sampling means 72 for checking purposes. It is moreover equipped with two channels 77a and 77b, which run along the side walls of the module and communicate with the whole of the ventilation and air distribution circuit and comprise means for intercommunication with the other channels having the same functions of the other modules of unit 10. This module also comprises an entrance lock 73 to control staff entrance and a hood 74 to control the air flow that circulates in the module, and in particular in area 75, where sampling is performed.

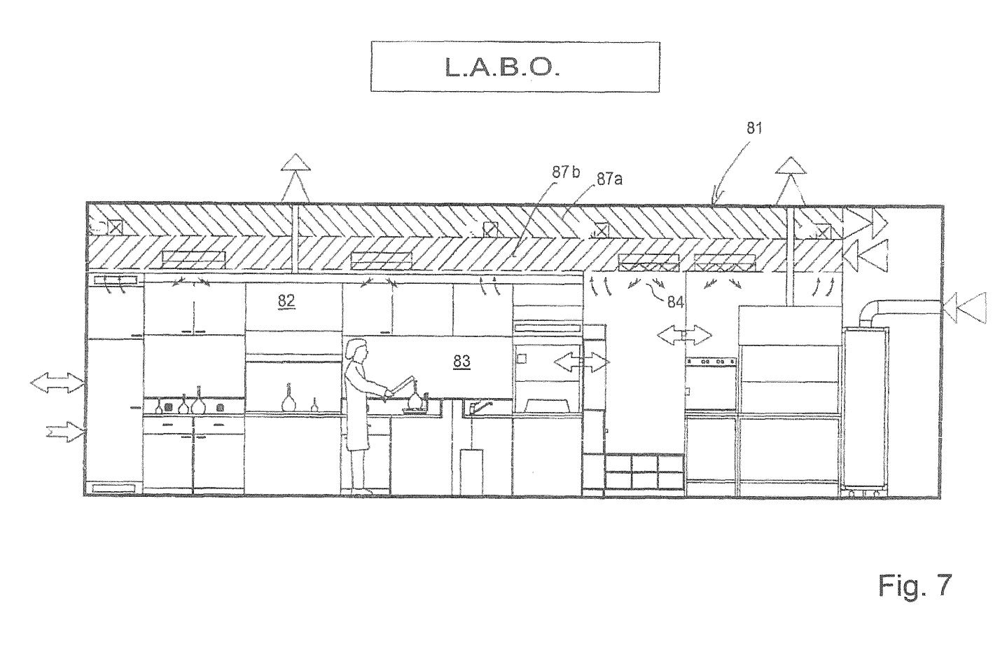

A second module 81, illustrated in FIG. 7, called laboratory module (L.A.B.O), belongs to this second group of control modules (C.O.N.T), and it is intended for chemical, physical and bacteriological analyses of the raw material (MA) samples previously isolated in module (P.R.E.L) and of the finished products. It includes three separated areas arranged inside of module 81, a bacteriological analyses area 82, a physical analyses area and a chemical analyses area 84, which are performed on the raw materials (MA) or on various products used within the framework of the method such as the purified water, the circulating air of the effluents. It is moreover equipped with two channels 87a and 87b, which run along the side walls of the module and communicate with the whole of the ventilation and air distribution circuit and comprise means for intercommunication with the other channels having the same functions of the other modules of unit 10.

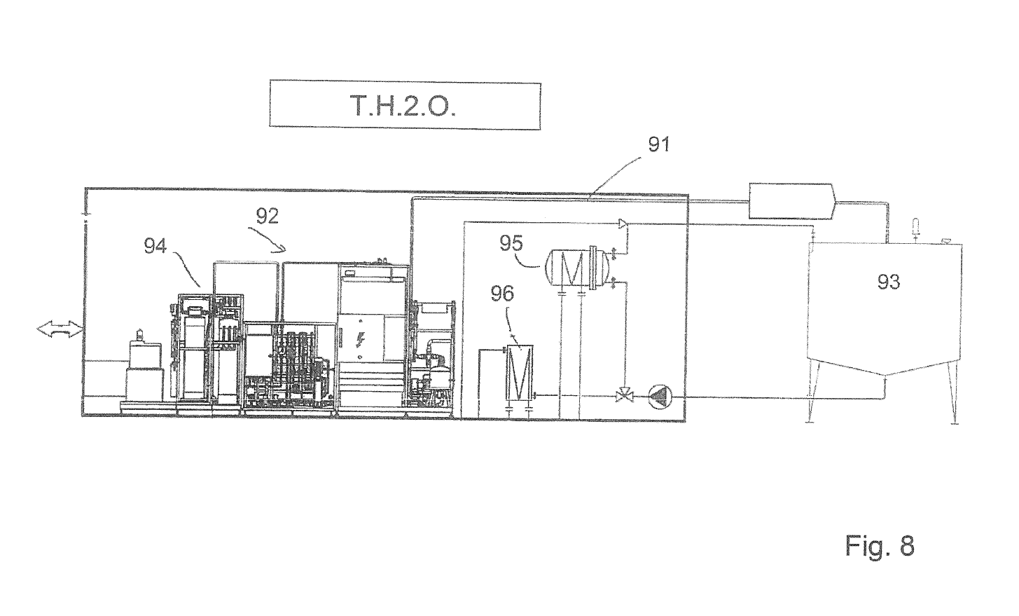

The first module of said third group 3, (F.L.U.X) is a so-called water treatment module 91 or (T.H.2.O) module illustrated in FIG. 8, whose function is to prepare the purified water in which the raw materials (MA) will be dissolved to produce the concentrates (CH) and to manage the refrigerated water and the hot water in two separate loops. It can for example include a reverse osmosis filtration machine 92 or the like. It includes a purified water storage tank 93 to allow having permanently a water reserve for use in the production phase. Moreover, module 91 can include a softener 94, a refrigerating exchanger 95 and a heating exchanger 96 in order to control independent hot water and refrigerated water circuits.

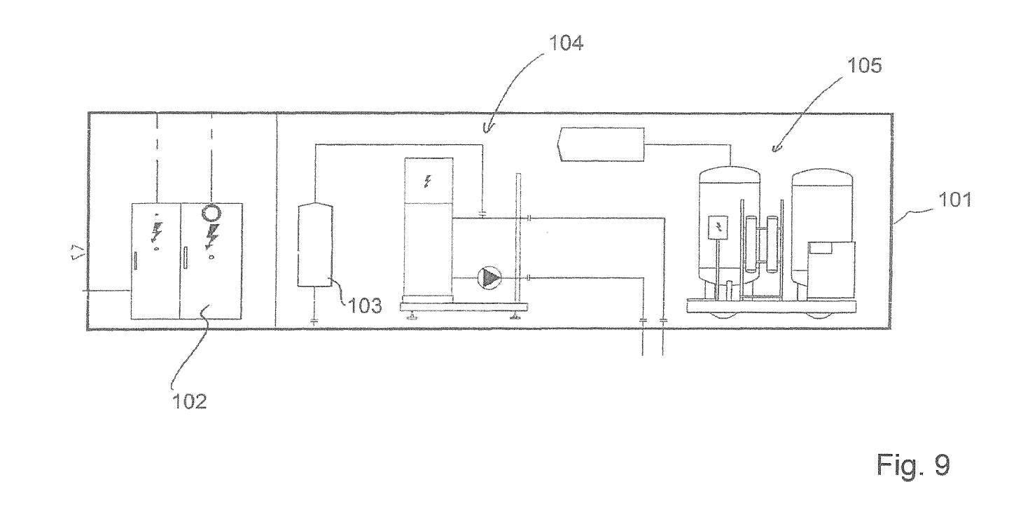

FIG. 9 illustrates the utilities module that is arranged for treating, preparing and conditioning flows and/or energies used in the course of the method. Module 101 can in particular comprise an electricity control unit 102, a softened water tank 103, a hot water production unit 104 and a compressed air generation unit 105.

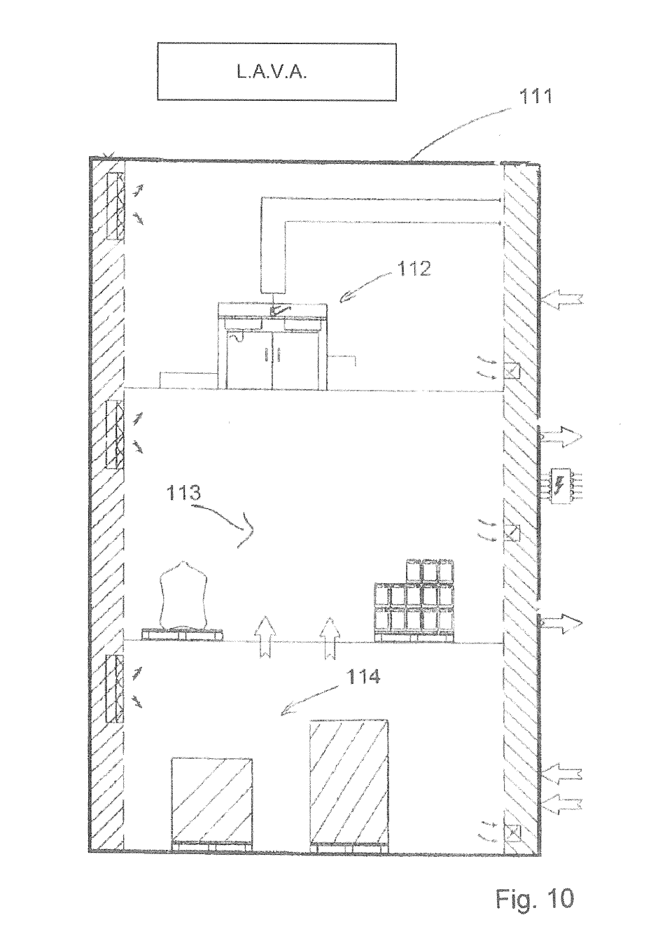

FIG. 10 illustrates washing module (LAVA) 111, which is provided with a washing room 112 to clean everything that enters in the treatment circuit of unit 10. Entrance locks 113 are provided to secure and control the whole of the entering material, as well as the operational staff. A storage area 114 under controlled atmosphere, advantageously under positive pressure and at a determined temperature, is provided in washing module (LAVA) 111.

FIG. 11 illustrates the effluents neutralization module called N.E.U.T, which belongs to group 4 (E.F.F.L), and whose function is to purge and neutralize the effluents before discharging them.

The modular design of this unit offers several advantages linked in particular to the constructive concept and to the distribution of all operational components, which are organized according to validated rules, immutable from one unit to another. The common centralized management of all flows and all energies constitutes a safety guarantee by allowing an overall view and generalized maintenance. The costs of an installation are foreseeable and do not depend on local parameters. The operational staff can be trained in any unit and the staff can be interchanged from one unit to another without requiring specific training.

The invention is not restricted to the examples of embodiment described and can present different aspects according to certain foreseeable evolutions or according to improvements of specific components. However, the scope of the invention is part of the framework defined by the claims.

1-18. (canceled)

19. A unit (10) for preparing at least one hemodialysis concentrate solution (CH), said solution comprising solid water-soluble materials comprising at least sodium chloride (NaCl), potassium chloride (KCl), calcium chloride (CaCl₂) and magnesium chloride (MgCl₂), including means for preparing said solution by introducing successively predetermined quantities of said solid water-soluble materials in a predetermined volume of purified water in a mixing container, means for carrying out individually a dosage control of said solid water-soluble materials introduced in said predetermined volume of the purified water and means for individually carrying out a compliance check of the concentrates solution obtained at an end of the process, wherein said unit comprises:
a sampling module (MPRE) to take at least a sample of each raw material to be dissolved in said volume of the purified water,
a laboratory module (MLAB) arranged to analyze a sample of each said raw material to control raw material compliance and quality,
a weighing module (MPES) to determine a quantity in weight of the raw material to be introduced in said predetermined volume of the purified water,
a solution preparation module (MPRE) to introduce and dissolve said quantity in weight of said raw material in said predetermined volume of the purified water, and
at least one module (MCON) for packaging the prepared hemodialysis concentrate solution.

20. The unit according to claim 19, wherein the unit comprises first analysis means to carry out, during a first intermediate step of an analysis phase of each sample of said raw materials, a quality check that comprises checking the compliance of the samples with a theoretical quality defined by at least one certificate specifying a nature and composition of the raw material concerned, as well as the compliance with regulations in force.

21. The unit according to claim 19, wherein the unit comprises second analysis means for carrying out, during a second intermediate step of an analysis phase of each sample of said raw materials, a purity check that comprises determining an actual weight of each of raw material which corresponds to a weight of a pure active substance contained in the raw material according to a respective purity of the raw material concerned.

22. The unit according to claim 19, wherein the unit comprises coupling means which interconnects all of the modules to perform respectively a centralized management of purified air flow that circulates through said modules, of liquid flow entering end exiting the various modules, of the pressurized gas flow, of the electrical energy flow used by said modules and circulation flow of workers and of components used during the method, in the course of said phases of the method and of the intermediate steps.

23. The unit according to claim 19, wherein the unit comprises a central purification equipment of the atmosphere that circulates through said modules and specific regulation means of air pressure contained in each of said respective modules, means for connecting the modules to said central purification equipment and means for interconnecting the modules to each other and/or to said central purification equipment.

24. The unit according to claim 23, wherein said means for connecting the modules to said central purification equipment comprises an air duct network arranged in a space located above a “false ceiling” reserved in a top section of each of said modules, said duct network comprising for each of said modules at least one inlet opening and at least one outlet opening, each of said inlet and outlet openings is equipped with a coupling valve to ensure an interconnection of at least one of said ducts of said duct network.

25. The unit according to claim 19, wherein the unit comprises central water treatment equipment that supplies at least a part of said modules, means for distributing treated water in each of said respective modules, means for collecting the waste water in each of said respective modules and means for connecting the modules to said central water treatment equipment and means for interconnecting the modules to each other and/or to said central water treatment equipment.

26. A method for preparing at least one hemodialysis concentrate solution, said solution comprising solid water-soluble materials comprising at least sodium chloride (NaCl), potassium chloride (KCl), calcium chloride (CaCl₂) and magnesium chloride (MgCl₂), wherein said solution being prepared by introducing successively, in a mixing container, predetermined quantities of said solid water-soluble materials in a predetermined volume of purified water contained in said mixing container, carrying out individually a dosage control of said solid water-soluble materials introduced in said predetermined volume of the purified water, and carrying out individually a compliance check of said concentrate solution for hemodialysis prepared by the method, said method comprising:
taking, during a sampling phase, a sample of each of said raw materials to be dissolved in said volume of purified water,
checking each sample of said raw material, during an analysis phase, to determine quality,
determining, during a weighing phase, a quantity in weight of said raw material to be introduced in said predetermined volume of the purified water,
introducing and dissolving, during a solution preparation phase, said quantity in weight of said raw material in said predetermined volume of the purified water, and
packaging, during a packaging phase, said prepared hemodialysis concentrate solution.

27. The method according to claim 26, wherein the analysis phase of each of said raw material samples to check the quality comprises a first intermediate step that comprises checking compliance of the samples with a theoretical quality defined by at least one certificate specifying a nature and composition of the raw material concerned.

28. The method according to claim 26, wherein the analysis phase of each sample of said raw materials to check quality comprises a second intermediate step including measurement of a purity of the raw material, which comprises determining an actual weight of each raw material that corresponds to a weight of an pure active substance contained within the raw material according to its respective purity.

29. The method according to claim 26, wherein said solution preparation phase to introduce and dissolve said quantity in weight of the raw material in said predetermined volume of purified water comprises a first intermediate step of introducing in said mixing container the quantity in weight of the raw material required to realize said hemodialysis concentrate solution, and the mixing container initially containing a fraction of said predetermined volume of purified water.

30. The method according to claim 29, wherein said fraction of said predetermined volume of purified water comprises at least approximately between 50 and 80% of said predetermined volume.

31. The method according to claim 29, wherein said solution preparation phase to introduce and dissolve said quantity in weight of said raw materials in said predetermined volume of purified water comprises a second intermediate step of adding in the mixing container, to the solution obtained after said first step, a remainder of said predetermined volume of purified water.

32. The method according to claim 26, wherein said packaging phase of said prepared hemodialysis concentrate solution comprises transferring, during a first intermediate step, said prepared hemodialysis concentrate solution into at least one storage tank.

33. The method according to claim 26, wherein said packaging phase of said prepared hemodialysis concentrate solution comprises packaging, during a second intermediate step, said prepared hemodialysis concentrate solution in at least one container arranged to supply a dialysis generator.

34. The method according to claim 26, wherein further comprising carrying out the phases of the method and of the intermediate steps in dedicated modules which are interconnected to perform a central management of the ambient air flows that circulate through said modules, of the liquid flow entering and exiting the various modules, of the pressurized gas flow, of the electrical energy flow used by the equipment of said modules and of the circulation flow of workers and of components used during the method, in the course of said phases of the method and of the intermediate steps.

35. The method according to claim 34, wherein, in order to perform a central management of the ambient air flow that circulate through said modules, one arranges an upper zone of each of said concerned modules, in an insulated space that contains circulation paths for an entering atmosphere and an exiting atmosphere, which are respectively in communication with a central air treatment module.

36. The method according to claim 34, wherein, in order to perform a central management of the liquid flow, connecting the purified water inlets and outlets of said modules concerned to each other and/or to an inlet and to an outlet of a central water treatment unit.
